(19) United States
(12) Patent Application Publication
(10) Pub. No.: US 2025/0025290 A1
(43) Pub. Date: Jan. 23, 2025

(54) MODULAR INTRAOCULAR IMPLANT FOR VITRECTOMY

(71) Applicant: Alcon Inc., Fribourg (CH)

(72) Inventor: Steven T. Charles, Memphis, TN (US)

(21) Appl. No.: 18/775,690

(22) Filed: Jul. 17, 2024

Related U.S. Application Data
(60) Provisional application No. 63/514,301, filed on Jul. 18, 2023.

(51) Int. Cl. A61F 2/16

(52) U.S. Cl. CPC A61F 2/1601; A61F 2/1648; A61F 2002/1681; A61F 2210/0076

(57) ABSTRACT

An apparatus for implant into an eye. The apparatus may comprise a receptacle configured to receive an optic; a flange circumscribing the receptacle, the flange configured to be disposed in the ciliary sulcus of the eye; and a fenestration through the flange, the fenestration configured to allow aqueous humor to pass anteriorly from the ciliary body to the trabecular meshwork of the eye. Some or all of the apparatus may have an oleophobic coating. For example, some embodiments of the flange may have an oleophobic coating. In some embodiments, the fenestration may be inferior to the receptacle. Additionally, or alternatively, the apparatus may further comprise at least two haptics coupled to the flange.

### PRIORITY CLAIM

This application claims the benefit of priority of U.S. Provisional Patent Application Ser. No. 63/514,301 titled “MODULAR INTRAOCULART IMPLANT FOR VITRECTOMY”, filed on Jul. 18, 2023, whose inventor(s) is/are Steven T. Charles, which is hereby incorporated by reference in its entirety as though fully and completely set forth herein.

### TECHNICAL FIELD

The invention set forth in the appended claims relates generally to ophthalmic implants, including, without limitation, modular intraocular implants for vitrectomy.

### BACKGROUND

The human eye can suffer a variety of maladies causing mild deterioration to complete loss of vision. While contact lenses and eyeglasses can compensate for some ailments, ophthalmic surgery may be required for others. For example, retinal detachment, traction retinal detachment, and trauma remain major causes of visual loss worldwide, despite continuing advances in vitreoretinal care, and pars plana vitrectomy is a leading management modality for the treatment for retinal detachment, traction retinal detachment and trauma.

While the benefits of vitrectomy and other ophthalmic surgical procedures are known, improvements to surgical systems, components, and processes can continue to improve outcomes and benefit patients.

### BRIEF SUMMARY

New and useful systems, apparatuses, and methods for eye surgery are set forth in the appended claims. Illustrative embodiments are also provided to enable a person skilled in the art to make and use the claimed subject matter.

For example, some embodiments comprise an apparatus for implant into an eye. The implant can provide a barrier to keep fluid from migrating into the anterior chamber, while micro-fenestrations can permit aqueous humor to pass from the ciliary body to the anterior chamber through the pupil and/or iridectomy in the inferior iris. Some embodiments may comprise an olcophobic coating and may be foldable to reduce requirements for incision size. Additionally, or alternatively, the implant may be configured to retain interchangeable optics.

More generally, an apparatus for implant into an eye may comprise a receptacle configured to receive an optic; a flange circumscribing the receptacle, the flange configured to be disposed in the ciliary sulcus of the eye; and a fenestration through the flange. The fenestration can be configured to allow aqueous humor to pass anteriorly from the ciliary body to the trabecular meshwork of the eye. In more particular embodiments, some or all of the apparatus may have an olcophobic coating. For example, some embodiments of the flange may have an olcophobic coating. In some embodiments, the fenestration may be inferior to the receptacle. Additionally, or alternatively, the apparatus may further comprise at least two haptics coupled to the flange.

In other embodiments, a method of inserting silicone oil into a vitreous chamber of an aphakic eye may comprise providing an apparatus comprising a receptacle configured to receive an optic, a flange circumscribing the receptacle, and one or more fenestrations through the flange. The apparatus may be placed between the iris of the eye and the vitreous chamber of the eye. The receptacle may be aligned with the pupil of the eye, and the fenestration oriented inferior to the pupil. The flange may be placed in the ciliary sulcus of the eye. Silicone oil may be injected into the vitreous chamber. The apparatus may be configured to prevent the silicone oil from migrating out of the vitreous chamber into the anterior chamber. For example, the receptacle can provide a fluid barrier between the vitreous chamber and the anterior chamber of the eye. Additionally, the fenestrations may be configured to allow aqueous humor to pass anteriorly from the ciliary body to the trabecular meshwork of the eye. In more particular embodiments, the method may further comprise fixating the haptics to the sclera of the eye.

In yet other embodiments, a method of treating an eye may comprise providing an apparatus comprising a receptacle, a flange circumscribing the receptacle, and a fenestration through the flange. The apparatus may be placed between the iris of the eye and the vitreous chamber of the eye. The receptacle may be aligned with the pupil of the eye, and the fenestration may be oriented inferior to the pupil. The flange may be placed in the ciliary sulcus of the eye. Silicone oil may be injected into the vitreous chamber, and a first optic may be inserted into the receptacle, wherein the first optic has a first optic power. The silicone oil may be removed from the vitreous chamber, and the first optic may be replaced with a second optic, the second optic having a second optic power. For example, the first optic power may be configured to compensate for the optical properties, such as the refractive index, of silicone oil, and the second optic power may be configured to compensate for optical properties of a different fluid, such as aqueous humor. In some embodiments, the method may be particularly advantageous for treating an aphakic eye.

Various embodiments, features, elements, and aspects described herein may be particularly advantageous for use in a vitrectomy. For example, some retina repair procedures use silicone oil to facilitate re-attachment, and many of these cases are aphakic. Some embodiments can provide a barrier to keep the silicone oil from migrating into the anterior chamber. Additionally, some embodiments may allow a temporary optic to be placed in the eye to compensate for the refractive index of the silicone oil while the retina heals. Once healed, the silicone oil may be removed, and the temporary optic may be replaced with a more permanent optic having a focal length appropriate for vitreous or aqueous humor.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is a front view of an example of an implant.

FIG. 2 is a schematic view of another example of the implant.

FIG. 3 is a front view of another example of the implant.

FIG. 4 is a schematic diagram of an example of the implant placed within an eye during or after a vitreoretinal surgery.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientations of various elements depicted in the attached drawings. In general, such relationships or orientations assume a frame of reference consistent with or relative to a patient in a position to receive an implant. However, as should be recognized by those skilled in the art, this frame of reference is merely a descriptive expedient rather than a strict requirement.

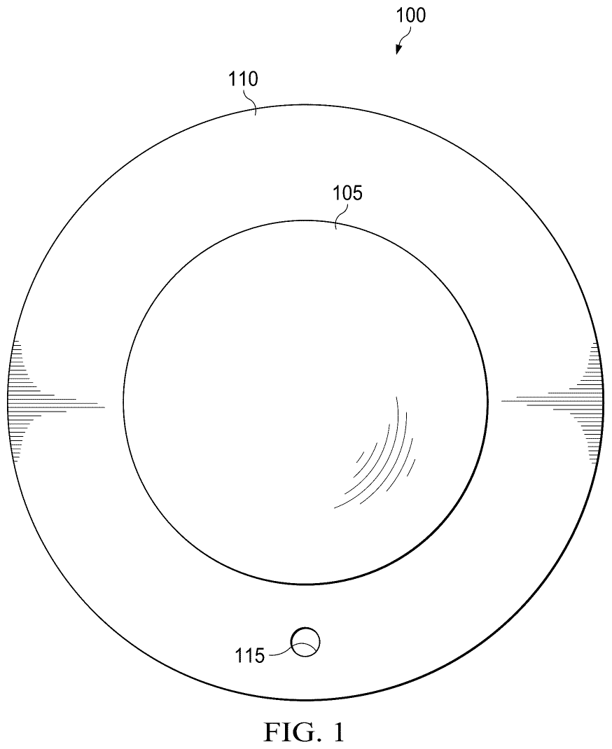

FIG. 1 is a front view of an example of an implant 100. As shown in the example of FIG. 1, some embodiments of the implant 100 may comprise a receptacle 105, a flange 110 circumscribing the receptacle 105, and at least one fenestration 115. In general, implant 100 is constructed of a flexible material such as acrylic, hydrophobic hydrogel, or PMMA. As shown in the example of FIG. 1, the flange 110 is generally circular, and the fenestration 115 is generally disposed in the peripheral portion of the implant 100. In more particular examples, the fenestration 115 is disposed in the flange 110. In some examples, the fenestrations may be micro-fenestrations having a width of about 0.5 microns to about 100 microns.

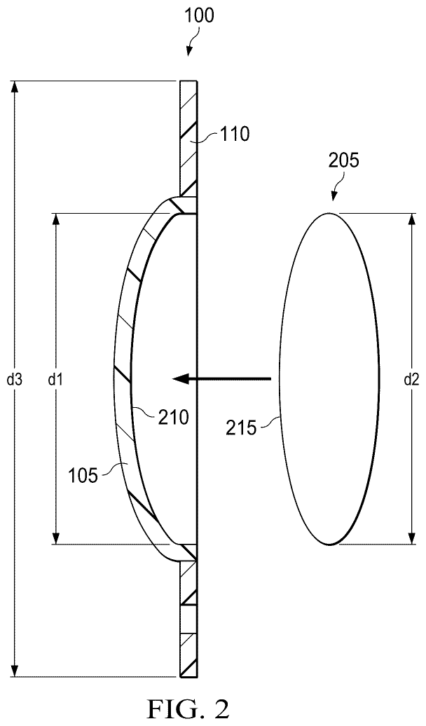

FIG. 2 is a schematic view of another example of the implant 100, illustrating additional details that may be associated with some embodiments. For example, FIG. 2 illustrates the receptacle 105 configured to receive an optic 205. The receptacle 105 may have an anterior surface 210 that is complimentary to a posterior surface 215 of the optic 205. The receptacle 105 generally has a diameter d1 that is larger than a diameter d2 of the optic 205. For example, the receptacle 105 may have a diameter d1 greater than about 5 millimeters to about 8 millimeters, and the optic 205 may have a diameter d2 that is in a range of about 5 millimeters to about 8 millimeters in some examples. Additionally, some examples of the flange 110 may have a diameter d3 that is suitable for placement in the ciliary sulcus. For example, a diameter d3 in a range of about 10 millimeters to about 14 millimeters may be preferable or advantageous.

In some embodiments, the receptacle 105 is configured to retain the optic 205. For example, the receptacle 105 may comprise a ridge or other retention features configured to mechanically attach the optic 205 to the receptacle 105 once inserted. Other examples of mechanical attachment may include mating or interlocking geometrical configurations corresponding to the receptacle 105 and the optic 205. Such geometrical configurations may be pre-formed by molding or cutting, for example, or formed in-situ with laser etching. Chemical attraction may be facilitated by using similar materials with a smooth surface finish activated by a surface treatment, for example.

In some embodiments, attachment mechanisms may be configured to have mating or interlocking geometries. Generally, such mechanisms may include a male portion and female portion that are releasably connectable. A female portion can be configured to receive the male portion and limit relative motion between the implant 100 and the optic 205 in at least two dimensions (e.g., superior-inferior and right-left). The female and male portions may be configured to have an interlocking geometry such that relative motion between the implant 100 and the optic 205 is limited in three dimensions (e.g., superior-inferior, right-left, anterior-posterior). An attachment mechanism may be engaged and disengaged by applying orthogonal force in a posterior (push) and anterior (pull) direction, respectively.

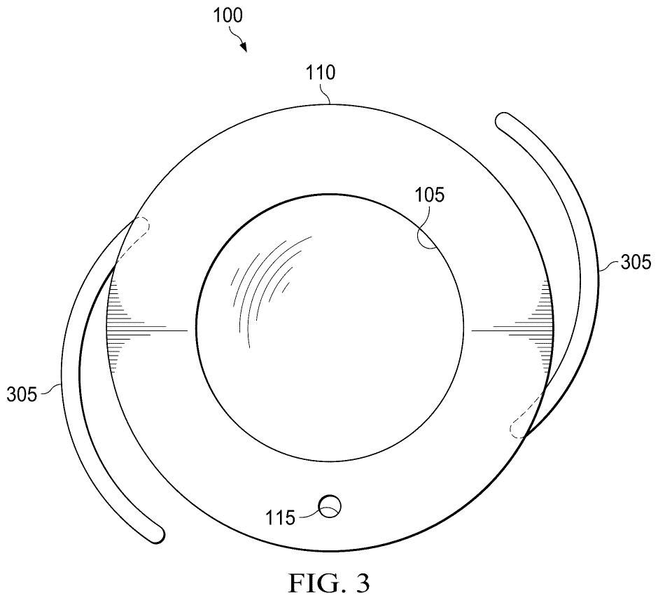

FIG. 3 is a front view of another example of the implant 100, illustrating additional details that may be associated with some embodiments. For example, some embodiments of the implant 100 may have one or more haptics 305. As illustrated in the example of FIG. 3, the haptics may be coupled to the flange 110 in some embodiments.

In general, components of the implant 100 may be coupled directly or indirectly. For example, the haptics 305 may be directly coupled to the flange 110 and may be indirectly coupled to the receptacle 105 through the flange 110. Coupling may include fluid, mechanical, or chemical coupling (such as a chemical bond), or some combination of coupling in some contexts. For example, the haptics 305 may be mechanically coupled to the flange 110. In some embodiments, components may also be coupled by virtue of physical proximity, being integral to a single structure, or being formed from the same piece of material. For example, the fenestration 115 may be coupled to the flange 110 by virtue of being integral to or formed with the flange 110.

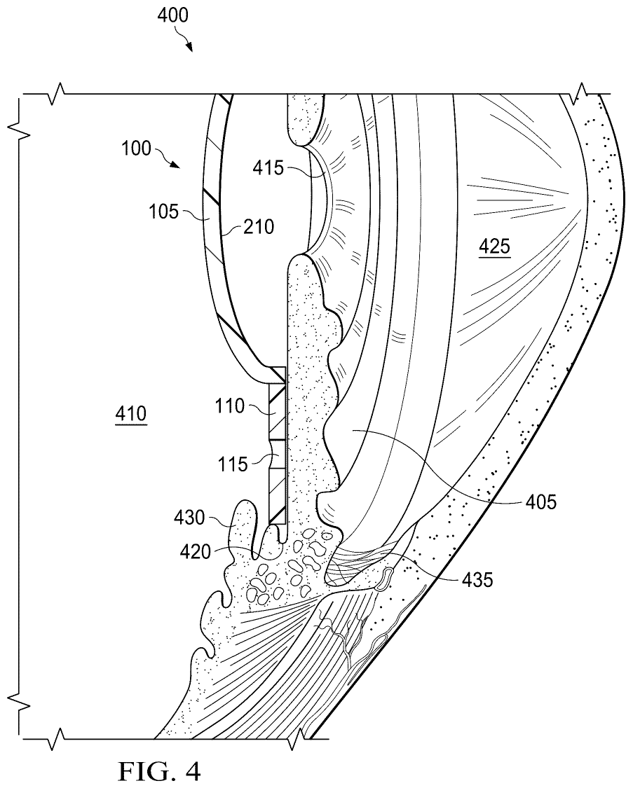

FIG. 4 is a schematic diagram of an example of the implant 100 placed within an eye 400 during or after a vitreoretinal surgery. The eye 400 in the example of FIG. 4 is aphakic, and the implant 100 is placed between the iris 405 of the eye 400 and the vitreous chamber 410 of the eye 400. In some examples, the implant 100 may be inserted through an incision in a folded configuration, and then unfolded in situ. The receptacle 105 may be aligned with the pupil 415 of the eye 400. In the example of FIG. 4, the fenestration 115 is oriented inferior to the pupil 415. The flange 110 of FIG. 4 is placed in the ciliary sulcus 420 of the eye 400. In other examples, the implant 100 may have a plurality of fenestrations disposed around the periphery of the flange 110. In some examples, the optic 205 (not shown in FIG. 4) may be inserted into the receptacle 105 so that the posterior surface 215 is in contact with the anterior surface 210.

Once placed in the eye 400 as illustrated in the example of FIG. 4, the receptacle 105 provides a fluid barrier between the vitreous chamber 410 and the anterior chamber 425 of the eye 400, and silicone oil or another tamponade may be injected into the vitreous chamber 410. The implant 100 can prevent the silicone oil from migrating out of the vitreous chamber 410. In other examples, the receptacle 105 may not provide a complete barrier. For example, the receptacle 105 may not be a solid surface, and the optic 205 may provide a barrier that complements the flange 110 to prevent silicone oil from migrating out of the vitreous chamber 410. Additionally, or alternatively, the fenestration 115 can be configured to allow aqueous humor to pass anteriorly from the ciliary body 430 to the anterior chamber 425 through the pupil 415 where the aqueous humor may pass through the trabecular meshwork 435 and be removed from the eye 400. Since the density of the silicone oil is less than the density of aqueous humor, the inferior position of the fenestration 115 can allow the aqueous humor to pass while the silicone oil floats posterior or superior to the fenestration 115. In some embodiments, an oleophobic coating on the implant 100 may be advantageous. For example, if silicone oil is injected into the vitreous chamber 410, an oleophobic coating on the flange 110 may be advantageous protecting the implant 100 from the silicone oil and/or preventing the silicone oil from diffusing into the optic 205.

In some embodiments, the haptics 305 may be fixated to the sclera of 435 the eye 400 to secure the implant 100 in position. For example, the haptics 305 may enable a Yamane flanged externalized haptics procedure in some embodiments. In alternative embodiments, the haptics 305 may be positioned in or proximate to other portions of the eye 400, such as in the ciliary sulcus 420.

While various tamponades have proven effective in improving outcomes after vitreoretinal surgery, some a susceptible to complications and it may be advantageous or necessary to remove the tamponade. For example, silicone oil can be used in complicated cases to increase pressure and facilitate retinal re-attachment, but silicone oil may emulsify and increase the risk of complications such as macular edema, glaucoma, and cataracts. Consequently, silicone oil should generally be removed from the vitreous chamber 410 after retinal re-attachment.

Silicone oil and other tamponades may have an index of refraction that is significantly different than aqueous humor or vitreous humor. Accordingly, the optic 205 may have a first optic power, and it may be advantageous or necessary to replace the optic 205 with a second optic (not shown) having a second optic power appropriate for the index of refraction associated with aqueous or vitreous humor.

The systems, apparatuses, and methods described herein may provide significant advantages. For example, many retina repair procedures may be aphakic, the capsule may be absent or disrupted, and/or zonules may be defective. Silicone oil can be an effective tamponade, particularly in extreme cases, but it is also associated with several notable complications, including increased risk of emulsification glaucoma and corneal endothelial damage, particularly if the silicone oil migrates into the anterior chamber. Embodiments of the implant 100 can significantly reduce such risks by providing a barrier to keep fluid from migrating from the vitreous chamber into the anterior chamber. Additionally, or alternatively, optics have not typically been used with silicone oil because the silicone oil is typically removed as soon as practical. Thus, patients usually have impaired vision for the duration of treatment. Additionally, optic capture in posterior capsulotomy can be surgically challenging and often not possible because of pseudo-exfoliation and other causes of defective zonules, such as Marfans syndrome. Significantly, some embodiments of the implant 100 may allow a temporary optic to be placed in the eye to compensate for the refractive index of a tamponade, which can restore vision almost instantly while the retina heals. Once healed, the tamponade may be removed, and the temporary optic may be replaced with a more permanent optic having a focal length appropriate for vitreous or aqueous humor. Some embodiments of the implant 100 may be particularly advantageous or useful if iris tissue is missing.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations, the optic 205 may be combined with the implant 100 or sold separately.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. An apparatus for implant into an eye having a ciliary body, a ciliary sulcus and an anterior chamber, the apparatus comprising:
a receptacle configured to receive an optic;
a flange circumscribing the receptacle, the flange configured to be disposed in the ciliary sulcus of the eye; and
a fenestration through the flange, the fenestration configured to allow aqueous humor to pass anteriorly from the ciliary body to the anterior chamber of the eye.

2. The apparatus of claim 1, wherein the flange has an oleophobic coating.

3. The apparatus of claim 1, wherein the fenestration is inferior to the receptacle.

4. The apparatus of claim 1, further comprising at least two haptics coupled to the flange.

5. The apparatus of claim 1, wherein the fenestration has a width in a range of about 0.5 microns to about 100 microns.

6. The apparatus of claim 1, wherein the apparatus comprises a plurality of fenestrations through the flange.

7. A method of inserting silicone oil into a vitreous chamber of an eye, the method comprising:
providing an apparatus comprising:
a receptacle configured to receive an optic,
a flange circumscribing the receptacle, and
a fenestration through the flange;
placing the apparatus between the iris of the eye and the vitreous chamber of the eye;
aligning the receptacle with the pupil of the eye;
orienting the fenestration inferior to the pupil;
placing the flange in the ciliary sulcus of the eye; and
injecting the silicone oil into the vitreous chamber.

8. The method of claim 7, wherein the apparatus is configured to prevent the silicone oil from migrating out of the vitreous chamber.

9. The method of claim 7, wherein the receptacle provides a fluid barrier between the vitreous chamber and the anterior chamber of the eye.

10. The method of claim 7, wherein the fenestration is configured to allow aqueous humor to pass anteriorly from the ciliary body to the trabecular meshwork of the eye.

11. The method of claim 7, wherein the flange has an oleophobic coating.

12. The method of claim 7, wherein the apparatus comprises at least two haptics coupled to the flange.

13. The method of claim 12, further comprising fixating the haptics to the sclera of the eye.

14. A method of treating an eye, the method comprising:
providing an apparatus comprising:
a receptacle,
a flange circumscribing the receptacle, and
a fenestration through the flange;
placing the apparatus between the iris of the eye and the vitreous chamber of the eye;
aligning the receptacle with the pupil of the eye;
orienting the fenestration inferior to the pupil;
placing the flange in the ciliary sulcus of the eye;
injecting silicone oil into the vitreous chamber;
inserting a first optic into the receptacle, the first optic having a first optic power;
removing the silicone oil from the vitreous chamber; and
replacing the first optic with a second optic, the second optic having a second optic power.

15. The method of claim 14, wherein the apparatus is configured to prevent the silicone oil from migrating out of the vitreous chamber.

16. The method of claim 14, wherein the receptacle provides a fluid barrier between the vitreous chamber and the anterior chamber of the eye.

17. The method of claim 14, wherein the fenestration is configured to allow aqueous humor to pass anteriorly from the ciliary body to the trabecular meshwork of the eye.

18. The method of claim 14, wherein the flange has an oleophobic coating.

19. The method of claim 14, wherein the apparatus comprises at least two haptics coupled to the flange.

20. The method of claim 19, further comprising fixating the haptics to the sclera of the eye.

21. The method of claim 20, wherein the eye is aphakic.

22. The method of claim 14, wherein the apparatus is configured to be inserted into the eye in a folded configuration.
